(19) United States
(12) Patent Application Publication
(10) Pub. No.: US 2012/0180554 A1
(43) Pub. Date: Jul. 19, 2012

(54) TOTAL ORGANIC CARBON (TOC) FLUID SENSOR

(75) Inventors: Richard Edward Leggett, Dickinson, TX (US); Vishnu Vardhanan Rajasekharan, Fort Collins, CO (US); Matthew Grant Collier, Longmont, CO (US)

(73) Assignee: HACH COMPANY, Loveland, CO (US)

(21) Appl. No.: 13/499,217

(22) PCT Filed: Sep. 29, 2010
(86) PCT No.: PCT/US10/50673

§ 371 Date: Mar. 29, 2012

Related U.S. Application Data
(60) Provisional application No. 61/248,037, filed on Oct. 2, 2009.

(51) Int. Cl. G01N 33/00

(52) U.S. Cl. 73/61.41

(57) ABSTRACT

A total organic carbon (TOC) fluid sensor (100) is provided according to an embodiment of the invention. The TOC fluid sensor (100) includes a first oxidization cell (101A), a second oxidization cell (101B), a gas permeable membrane (106) configured to allow carbon dioxide to equilibriate between the first oxidization cell (101A) and the second oxidization cell (101B), a first conductivity sensor (136A), and a second conductivity sensor (136B). The TOC fluid sensor (100) oxidizes a fluid portion in the first oxidization cell (101A) to create carbon dioxide, equilibriates the carbon dioxide between the first oxidization cell (101A) and the second oxidization cell (101B), obtains a second cell conductivity information, and determines a TOC quantity in the fluid under test from the second cell conductivity information when the first cell oxidization is substantially complete.

### BACKGROUND OF THE INVENTION

1. Field of the Invention

The invention is related to the field of carbon sensors, and in particular, to aqueous carbon sensors.

2. Statement of the Problem

The usefulness of water often depends on how clean the water is. Water suitable for washing may not be suitable for drinking. Water suitable for drinking may not be suitable for manufacturing drugs for oral medications or for injection. Thus, standards of cleanliness have been established for each type of use.

Because carbon is a common element present in all plants and animals, the measurement of Total Organic Carbon is an important measurement to assess the cleanliness of water, used from carbon levels of less than μg/Cl to many thousands of mgC/l. The carbon content extends from ultra pure water (UPW) at a low level to up to an industrial waste water (IWW) level.

UPW is used as a raw material, ingredient, and solvent in the processing, formulation and manufacture of products in life sciences industry. This industry mandates water regulations. Europe, Japan, United States and China have published official documents listing drugs with directions for specific quality attributes. These publications are known as pharmacopoeia. Pharamcopoeial standards regulate water grades and give specific quality parameters and test procedures. TOC and conductivity are two of the parameters which fall under the Pharamacopoeial regulatory mandate. Life sciences industry is interested in using TOC as a surrogate method to validate the cleaning protocol of vessels used to manufacture the drugs and other products. The sample matrix encountered in this application is more complex and has a higher conductivity than point of use applications.

UPW is particularly important in the semiconductor wafer fabrication industry, where the water used in repetitive operations must not contain impurities. UPW is consequently used to avoid unwanted interstitial molecular structures being created within the semiconductor lattices, thus lowering the yield of semiconductor product. TOC monitoring of UPW has been traditionally been used to ensure that the water is indeed clean enough to be used in these operations. However, TOC analyzers only measure carbon, and recently semiconductor companies have expressed the wish to measure other species which would normally be classified as interferences to the measurement of TOC.

There is a need, therefore, for detecting interference materials in a fluid under test when measuring carbon content.

### ASPECTS OF THE INVENTION

In one aspect of the invention, a total organic carbon (TOC) fluid sensor (100) comprises:
a first oxidization cell and a second oxidization cell that receive a fluid under test;
a gas permeable membrane configured to allow carbon dioxide to equilibriate between the first oxidization cell and the second oxidization cell;
a first conductivity sensor configured to measure a first oxidization cell conductivity; and
a second conductivity sensor configured to measure a second oxidization cell conductivity;
wherein the TOC fluid sensor is configured to oxidize a fluid portion to produce carbon dioxide gas, equilibriate the carbon dioxide gas between the first oxidization cell and the second oxidization cell, obtain a second cell conductivity information, and determine a TOC quantity in the fluid under test from the second cell conductivity information when the first cell oxidization is substantially complete.

Preferably, the second oxidization cell is substantially free of interference materials that may exist in the first oxidization cell as a result of the equilibriation.

Preferably, determining the TOC quantity further comprises determining the TOC quantity from a first cell conductivity information and the second cell conductivity information when the first cell oxidization is substantially complete.

Preferably, the first cell oxidization is substantially complete when the first cell conductivity information becomes substantially unchanging.

Preferably, the first cell oxidization is substantially complete when the first cell conductivity information and the second cell conductivity information both become substantially unchanging.

Preferably, the oxidization can be performed in both the first oxidization cell and in the second oxidization cell in the absence of any interference materials.

Preferably, the TOC fluid sensor is further configured to detect interference materials in the fluid under test if a first cell conductivity increase is greater than a second cell conductivity increase.

Preferably, the TOC fluid sensor is further configured to quantify interference materials in the fluid under test using the first cell conductivity information and the second cell conductivity information, with the quantifying characterizing a carbon atom quantity and a non-carbon atom quantity using a ratiometric analysis of the first oxidization cell conductivity and the second oxidization cell conductivity.

Preferably, the TOC fluid sensor is further configured to introduce a carrier gas that is substantially void of carbon dioxide into the first oxidization cell, with the carrier gas substantially stripping the carbon dioxide, and quantify interference materials in the fluid under test using the first cell conductivity information.

Preferably, further comprising a pump that re-circulates the fluid in the first oxidization cell.

In one aspect of the invention, a total organic carbon (TOC) fluid measurement method comprises:
oxidizing a fluid portion of a fluid under test in a first oxidization cell to create carbon dioxide;
equilibriating the carbon dioxide between the first oxidization cell and a second oxidization cell;
obtaining a first cell conductivity information and a second cell conductivity information; and
determining a TOC quantity in the fluid under test from the second cell conductivity information when the first cell oxidization is substantially complete.

Preferably, determining the TOC quantity further comprises determining the TOC quantity from a first cell conductivity information and the second cell conductivity information when the first cell oxidization is substantially complete.

Preferably, the first cell oxidization is substantially complete when the first cell conductivity information becomes substantially unchanging.

Preferably, the first cell oxidization is substantially complete when the first cell conductivity information and the second cell conductivity information both become substantially unchanging.

Preferably, the oxidization can be performed in both the first oxidization cell and in the second oxidization cell in the absence of any interference materials.

Preferably, further comprising detecting interference materials in the fluid under test if a first cell conductivity increase is greater than a second cell conductivity increase.

Preferably, further comprising quantifying interference materials in the fluid under test using the first cell conductivity information and the second cell conductivity information, with the quantifying characterizing a carbon atom quantity and a non-carbon atom quantity using a ratiometric analysis of the first oxidization cell conductivity and the second oxidization cell conductivity.

Preferably, further comprising introducing a carrier gas that is substantially void of carbon dioxide into the first oxidization cell, with the carrier gas substantially stripping the carbon dioxide and quantifying interference materials in the fluid under test using the first cell conductivity information.

Preferably, further comprising re-circulating the fluid in the first oxidization cell during at least a portion of the oxidizing.

### DESCRIPTION OF THE DRAWINGS

The same reference number represents the same element on all drawings.

FIG. 1 shows a total organic carbon (TOC) fluid sensor according to an embodiment of the invention.

FIG. 2 is a flowchart of a TOC fluid measurement method according to the invention.

FIG. 3 shows the TOC fluid sensor according to another embodiment of the invention.

FIG. 4 shows the TOC fluid sensor according to another embodiment of the invention.

### DETAILED DESCRIPTION OF THE INVENTION

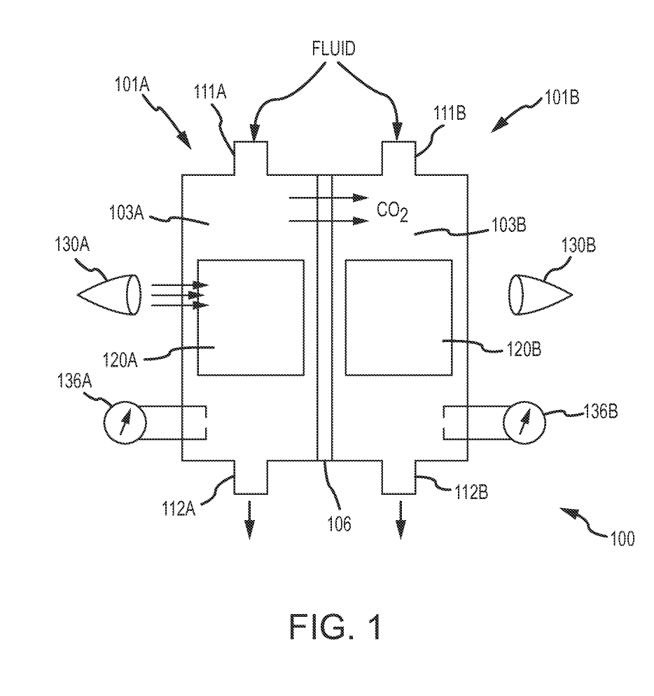

FIGS. 1-3 and the following description depict specific examples to teach those skilled in the art how to make and use the best mode of the invention. For the purpose of teaching inventive principles, some conventional aspects have been simplified or omitted. Those skilled in the art will appreciate variations from these examples that fall within the scope of the invention. Those skilled in the art will appreciate that the features described below can be combined in various ways to form multiple variations of the invention. As a result, the invention is not limited to the specific examples described below, but only by the claims and their equivalents.

FIG. 1 shows a total organic carbon (TOC) fluid sensor 100 according to an embodiment of the invention. The TOC fluid sensor 100 quantifies carbon in a fluid under test. The fluid under test may comprise water, a fluid including water, or may comprise other fluids. The TOC fluid sensor 100 operates by oxidizing carbon in the fluid under test to produce carbon dioxide gas, and then quantifies the produced carbon dioxide gas.

The TOC fluid sensor 100 is reagentless. No reagents are needed by the TOC fluid sensor 100 for oxidization. No reagents are needed by the TOC fluid sensor 100 for TOC quantification. No reagents are needed by the TOC fluid sensor 100 in order to detect or quantify interference materials.

At a most basic level, the TOC fluid sensor 100 operates on a first fluid portion of the fluid under test and a second fluid portion. The TOC fluid sensor 100 according to the invention is generally operated so as to oxidize the first fluid portion but not the second fluid portion. The oxidization process converts carbon to carbon dioxide gas. The produced carbon dioxide gas is allowed to equilibriate between the two fluid portions, with the first fluid portion now comprising the oxidized fluid portion and some of the produced carbon dioxide gas. The second fluid portion, after the equilibriation, now comprises an un-oxidized fluid portion and some of the produced carbon dioxide gas. The difference is that the change in the second fluid portion is due only to the equilibriation (i.e., migration) of the produced carbon dioxide gas from the first fluid portion and into the second fluid portion. Consequently, the change in the second fluid portion is entirely due to the carbon dioxide equilibriation. This change in the second fluid portion is quantified in order to quantify the carbon content of the fluid under test. The quantification requires that the division of the produced carbon dioxide gas be compensated for in order to generate a TOC level, however.

Advantageously, the TOC fluid sensor 100 produces an accurate TOC level. The TOC fluid sensor 100 produces a TOC level that is not affected by the presence of interference materials in the fluid under test. The TOC fluid sensor 100 produces a TOC level that does not include any of the interference materials. As a result, the TOC fluid sensor 100 produces a much more accurate TOC quantification value. As a result, the TOC fluid sensor 100 produces a TOC quantification value that reflects only the carbon present in the fluid under test.

Another advantage is that the TOC fluid sensor 100 may also detect interference materials in the fluid under test, in addition to generating a TOC level. In some cases, the TOC fluid sensor 100 may even quantify the types and/or amounts of interference materials present in the fluid under test, such as where variations in the composition of the fluid under test are known (such as the occasional presence of residual additives in treated drinking water, for example). Consequently, the expected variations may be detected, or even detected in substantially real time in some applications.

The TOC fluid sensor 100 comprises at least a first cell 101A and a second cell 101B. Each cell 101A, 101B may comprise a partial or complete reaction chamber. Each cell 101A, 101B includes a chamber 103A, 103B for receiving a portion of the fluid under test. The chambers 103A, 103B may be identical in size and/or shape or may differ. The two cells 101A, 101B can receive a fluid under test, wherein the measurement produces a quantification of a total organic carbon (TOC) in the fluid.

The cells 101A, 101B in the embodiment shown each include a fluid inlet 111A, 111B and a fluid outlet 112A, 112B. However, it should be understood that the cells 101A, 101B can share inlets and/or outlets. Each cell can alternatively have a single port (or single shared port between the two) that is used for inputting and outputting fluid.

The first cell 101A and the second cell 101B share a gas permeable membrane 106 that separates the two chambers 103A, 103B. The membrane 106 comprises a gas permeable membrane 106 in some embodiments, wherein the membrane 106 allows gas to pass therethrough. If oxidization is performed in the first cell 101A, for example, then any produced carbon dioxide gas will be able to equalize between the two cells 101A, 101B. Consequently, when oxidization is performed in the first oxidization cell 101A, at least some of the produced carbon dioxide will migrate through the gas permeable membrane 106 into the second oxidization cell 101B. It should be understood that the carbon dioxide produced in the first oxidization cell 101A will equilibrate between the two cells, with the carbon dioxide produced in the first oxidization cell 101A, for example, migrating to the second oxidization cell 101B until equal gas pressures are achieved. The gas migration/equilibriation occurs even though the oxidization is performed only in the first oxidization cell 101A. The two chambers 103A, 103B will have substantially equal gas pressures when the equilibriation is complete.

Approximately half of the produced carbon dioxide gas will migrate to the second cell 101B (assuming that the oxidization cells 101A, 101B are of approximately equal size). Quantification of carbon dioxide in the second cell 101B will enable a total carbon quantification, wherein the total carbon will be twice that as indicated/measured due to the carbon dioxide present in the second cell 101B (where the two cells are equal in size). If the two cells are unequal in size, then the carbon dioxide in the first cell 101A may be found by multiplying the second cell conductivity measurement by a relative size/volume fraction, for example.

The oxidization cells 101A, 101B may include oxidizing materials 120A, 120B. It should be understood that oxidizing materials are not required in both cells, but may be included in both cells for flexibility of use and for cleaning the cells, for example.

The oxidizing materials 120A, 120B can comprise any manner of oxidizing materials. In one embodiment, the oxidizing materials 120A, 120B comprise titanium dioxide (TiO₂) films, film portions, deposits, inserts, or so forth, that require the oxidizing materials to be radiated with ultraviolet (UV) light in the presence of the fluid under test. However, other oxidizing materials 120A, 120B or oxidizing systems are contemplated and are within the scope of the description and claims.

The oxidization cells 101A, 101B may include light sources 130A, 130B. Light generated by the light sources 130A, 130B can be directed onto the oxidizing materials 120A, 120B in order to oxidize carbon in a fluid under test. The light sources 130A, 130B may be built into the interior of the oxidization cells 101A, 101B in some embodiments. Alternatively, the light sources 130A, 130B may be external to the chambers 103A, 103B and positioned to direct light into the oxidization cells 101A, 101B. It should be understood that light sources are not required in both cells, but may be included in both cells for flexibility of use and for cleaning the cells, for example.

In some embodiments, the light sources 130A, 130B are capable of generating ultraviolet (UV) light. However, light (or electromagnetic radiation) of any suitable wavelength may be generated and used. The light sources 130A, 130B may comprise incandescent, semiconductor, laser, fluorescent, or other light sources suitable for illuminating the oxidizing materials 120A, 120B. The light sources 130A, 130B can comprise point light sources, line light sources, cylindrical light sources, or other configurations. The light sources 130A, 130B can include light pipes, optical fibers, or any manner of light transmission components used to provide light to the oxidizing materials 120A, 120B. The light sources 130A, 130B can further include diffusers, lenses, shutters, filters, grates, masks, or any other optical components useful for directing and/or controlling light and providing light to the oxidizing materials 120A, 120B when needed.

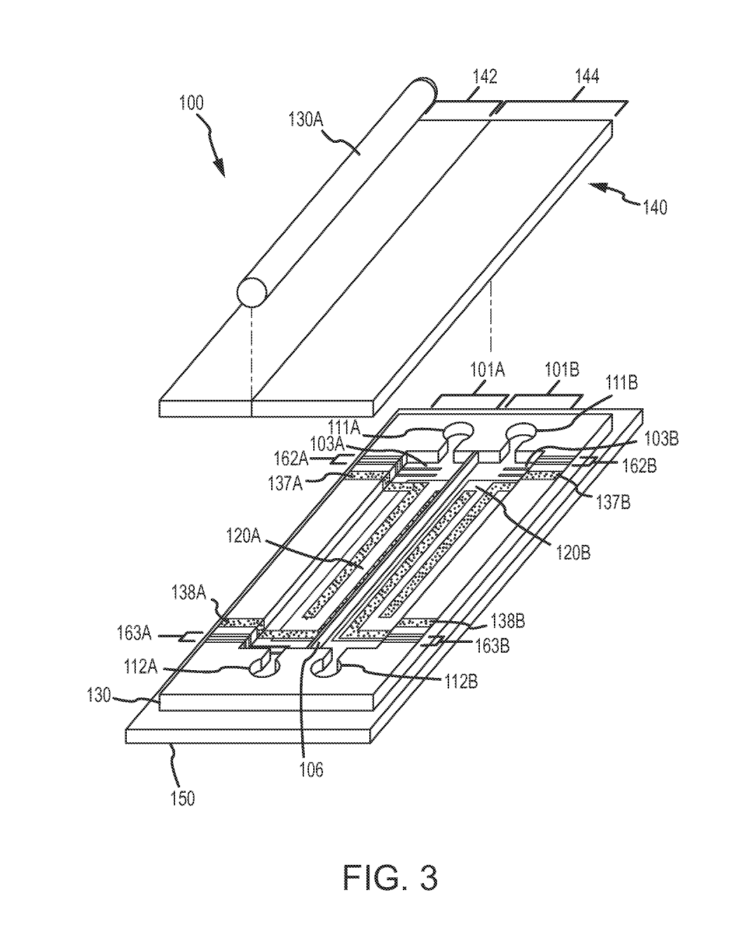

The oxidization cells 101A, 101B may include one or more conductivity sensors 136A, 136B. In the embodiment shown, the quantification apparatus comprises two conductivity measurement devices 136A, 136B that independently measure the respective conductivity in the first and second cells 101A, 101B. Each conductivity sensor can include electrodes extending into a chamber 103A, 103B and in contact with the fluid therein (see electrode pair 137A, 138A and electrode pair 137B, 138B of FIG. 3). In some embodiments, an oxidizing material (such as titanium dioxide, TiO₂, for example) can also be used as electrodes for the conductivity measuring devices 136A, 136B.

An increase in fluid conductivity is known to indicate an increase in carbon dioxide in the fluid. As a result, a change in the fluid conductivity can be used to quantify the carbon dioxide (and therefore the carbon) in the fluid.

A conductivity difference will exist if the first oxidization cell 101A includes fluid, produced carbon dioxide gas, and interference materials, while the second oxidization cell 101B includes only fluid and produced carbon dioxide gas, with no interference materials. By comparing the conductivity information measured in the first oxidization cell 101A to the conductivity information measured in the second oxidization cell 101B, interference materials can therefore be detected.

However, an increase (or change) in conductivity is also known to indicate other conductive materials in the fluid. These materials may be characterized as being interference materials for interfering with the conductivity of the fluid under test. An interference material, by affecting a conductivity characteristic of the fluid, may interfere with the carbon quantification. As a result, quantification of carbon using conductivity information may produce inaccurate or even invalid results when interference materials are present.

The interference materials may comprise ionic compounds. The interference materials may include interference products that are at least partially produced by the oxidation process. Such oxidization by-products can lead to erroneous conductivity readings. Alternatively, the interference materials may comprise materials already in the fluid under test, such as urea or other contaminants, for example.

If the conductivity of the first oxidization cell 101A is substantially equal to the conductivity of the second oxidization cell 101B, then there are no interference materials in present in the first oxidization cell 101A (assuming that the two cells are the same and therefore the produced carbon dioxide is equal in both cells). Conversely, if the conductivity of the first oxidization cell 101A is substantially different from the conductivity of the second oxidization cell 101B, then interference materials are present in the first oxidization cell 101A (again, assuming that the two cells are the same and therefore the produced carbon dioxide is equal in both cells).

The TOC fluid sensor 100 is capable of detecting interference materials in the fluid under test. The detection may include detecting one or more interference materials. As a result, the TOC fluid sensor 100 can generate a notification or alarm, can record the presence or absence of interference materials, or any other desired action related to detection of interference materials in the fluid under test.

The TOC quantification value can comprise a carbon mass or carbon density (i.e., a mass of carbon per volume of fluid) for the fluid under test. The TOC quantification value can comprise a carbon volume in relation to the volume of fluid under test. The TOC quantification value can comprise a carbon molar count in relation to a fluid molar count. It should be understood that the above are examples given merely for illustration. Additional processes or quantification types that are capable of quantifying produced carbon dioxide in relation to the fluid the carbon dioxide is produced from are contemplated and are within the scope of the description and claims.

The fluid under test can be any manner of fluid, such as water, for example. Alternatively, the fluid under test can partially comprise water. However, the fluid under test is not limited to water or any particular percentage of water, and the TOC fluid sensor 100 may be used with other fluids.

In the TOC fluid sensor, two oxidation cells are coupled via a membrane. During analysis, oxidation of a fluid portion is performed in only one of the two cells. Interference materials present in the fluid under test, or created during the oxidation, are retained in the oxidizing cell. The interference materials in the fluid under test cannot cross the membrane. In contrast, the carbon dioxide created by the oxidation is equilibrated between the two oxidization cells via the membrane 106. As a result, the carbon dioxide becomes evenly distributed between the two cells while any interference materials remain strictly in the oxidizing cell. Consequently, the non-oxidizing cell may be used to measure a TOC level, with the resulting TOC quantification comprising a quantification that does not include any quantification of interference materials. However, a TOC level in the non-oxidizing cell is not the total amount of carbon dioxide, as the produced carbon dioxide is present in both cells due to the equilibriation. The equilibriation process can be subsequently accounted for, as the carbon dioxide will be substantially equal in pressure after the equilibriation. As a result, the amount in each cell may be determined according to the relative volumes of the two cells. If the second oxidization cell 101B is identical in volume to the first oxidization cell 101A, then the amount of carbon dioxide subsequently quantified in the first oxidization cell 101A will be one-half of the total carbon dioxide produced during the oxidization.

Further, the differences between the two oxidization cells can be used to detect interference materials. Moreover, the differences between the two oxidization cells may be used to quantify an amount of interference materials in the fluid.

In operation, the TOC fluid sensor 100 may be operated to oxidize the first fluid portion to produce carbon dioxide gas, equilibriate the carbon dioxide gas between the first oxidization cell 101A and the second oxidization cell 101B, obtain a second cell conductivity information, and determine a TOC quantity in the fluid under test from the second cell conductivity information when the first cell oxidization is substantially complete.

In some embodiments, the TOC fluid sensor 100 may be operated wherein a carrier gas that is substantially void of carbon dioxide is introduced into the first oxidization cell 101A. The carrier gas may be introduced after the first cell oxidization process is substantially complete and any produced carbon dioxide has been substantially equilibriated. The carrier gas may be used to substantially strip the carbon dioxide produced during or after the oxidation. As a result, the conductivity remaining in the first oxidization cell 101A after the stripping process will be solely due to the interference materials, if present.

After the TOC level measurement has been obtained, the second oxidization cell 101B may be subjected to an oxidization process in order to clean the second oxidization cell 101B before any subsequent use.

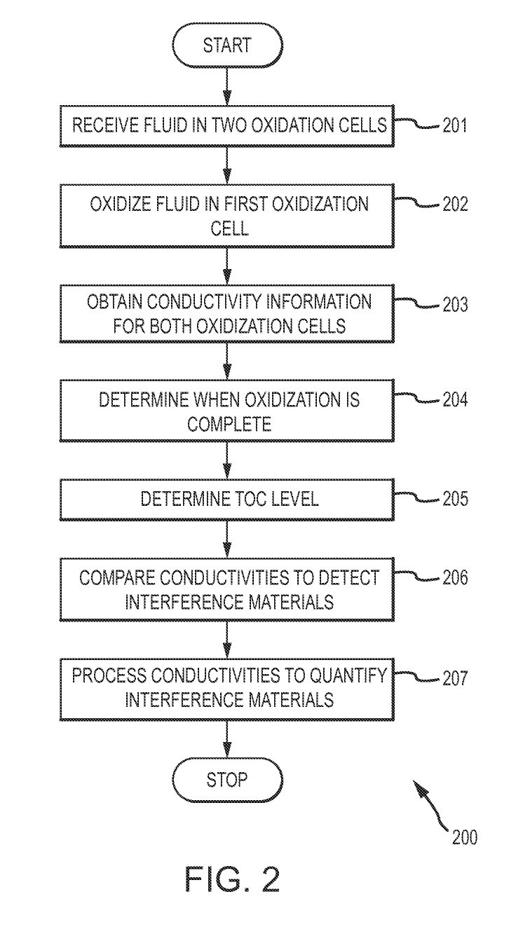

FIG. 2 is a flowchart 200 of a TOC fluid measurement method according to the invention. The TOC fluid measurement method is reagentless. No reagents are needed for oxidization. No reagents are needed for TOC quantification. No reagents are needed in order to detect or quantify interference materials. In step 201, a fluid under test is received in the oxidization cells of a TOC fluid sensor. The cells may be substantially similar or identical in construction and in size, or may differ.

In step 202, the first fluid portion in the first oxidization cell is oxidized. The oxidization may be done in only one of the two oxidization cells. However, it should be understood that either cell may be subject to the oxidization process. The oxidization may include at least partially illuminating an oxidizing material in the first oxidization cell with ultraviolet (UV) light (i.e., photo-catalytic oxidization). The UV light, acting on the oxidizing material, initiates and sustains the oxidization process. As a result of the oxidization, any carbon in the fluid will be oxidized and will be substantially converted into carbon dioxide (CO₂) gas. The carbon dioxide gas will pass through the common gas permeable membrane and equilibrate between the two oxidization cells. As a result, equal portions of carbon dioxide will be present in the two oxidization cells. However, due to the gas permeable membrane, no interference materials produced by the oxidization (or otherwise present in the fluid) will migrate to the second oxidization cell.

In step 203, conductivity information for both oxidization cells may be obtained and recorded. Generally, the conductivity of both cells is monitored and recorded before, during, and after the oxidization process. This may include generating a conductivity profile that reflects the conductivity levels in the cell, as well as rate of change of conductivity in the cell.

A second cell conductivity increase will occur as a result of the oxidization process and the resulting carbon dioxide equilibriation. The second cell conductivity increase will therefore comprise an increase in fluid conductivity as a result of the carbon dioxide equilibriating into the second oxidization cell.

A first cell conductivity increase will be at least as large as the second cell conductivity increase. However, it should be understood that the first cell conductivity increase will be larger than that of the second cell if interference materials are created by the oxidization. As a result, the two conductivity measurements may be larger than the conductivity increase due to just the quantity of TOC oxidized in the fluid under test.

In step 204, the end of the oxidization process is determined. If the oxidization process is determined to be substantially complete, then the oxidizing source(s) are de-energized or taken offline. It is desirable that the fluid under test is completely oxidized in order to convert all carbon into carbon dioxide gas. Depending on the amount and conductivities of short lived radical species produced during the oxidation, the conductivity may rise or fall immediately after the oxidation source(s) are switched off. A complete oxidation ensures that all the organics are accounted in the TOC measurement. Some regulatory bodies in the life sciences industry require the complete oxidation of the organics in the TOC measurement.

It is known that carbon dioxide in a fluid such as water, or a solution including water, will exhibit an increase in conductivity when the carbon is oxidized into carbon dioxide gas. Therefore, the end of the oxidization process may be determined by monitoring the conductivity in the first oxidization cell. When the conductivity stops changing, i.e., when the conductivity reaches a substantially stable state, then the fluid has been substantially completely oxidized.

The equilibrium conductivity indicates the end point of the oxidization process. The initial conductivity before oxidization, the increase in conductivity and the equilibrium conductivity during oxidization, and the stable conductivity after oxidization in both cells comprise the full conductivity profile in some embodiments.

Alternatively, or in addition, the conductivity of the second oxidization cell may be monitored in order to detect the completion of oxidization in the first oxidization cell. Because of the gas permeable membrane between the two oxidizing cells, carbon dioxide gas produced in the first oxidization cell will equilibriate between the two oxidization cells, as has been previously discussed. As a result, the conductivity in the second oxidization cell will also increase due to an increase in carbon dioxide gas. Therefore, when the rate of conductivity change in the second oxidization cell reaches a stable state (the rate of change in conductivity approaches zero), then the oxidization process in the first oxidization cell is substantially complete. In a third alternative, both the first cell conductivity and the second cell conductivity may be monitored to determine the end of the oxidization process.

In step 205, the TOC level in the fluid under test is determined from the second cell conductivity information. As was previously noted, the second cell conductivity information includes conductivity levels and includes the rate of conductivity change over time and may comprise a conductivity profile. The TOC level is obtained from the second cell conductivity information when the conductivity has reached an equilibrium state. This equilibrium conductivity can be calculated from the raw conductivity measurements or can be mathematically derived as the first or second derivative of the raw conductivity measurement. The TOC level can be derived from the second cell conductivity information using a linear or non-linear equation, from a table or other data structure, or in any other suitable fashion. Advantageously, the TOC level derived from the second cell conductivity information does not include any quantification of interference materials. The change in conductivity in the second oxidization cell is due only to the equilibriated carbon dioxide gas.

The TOC level quantification may be expressed as a produced carbon dioxide mass. The TOC level quantification may be expressed as a produced carbon dioxide volume. The TOC level quantification may be expressed as a number of moles of produced carbon dioxide.

However, the conductivity change in the second oxidation cell does not necessarily reflect the total carbon dioxide gas produced unless the two oxidization cells are substantially equal in volume. Where the two cells are identical, the TOC level determined from the second cell conductivity information may be doubled in order to generate the overall TOC level for the fluid under test. Where the two oxidization cells are not equal, a volumetric multiplier or ratio may be used to generate the final TOC level from the second cell conductivity information.

In another alternative, if it is determined that no interference materials were produced as a result of the oxidization in the first oxidization cell, then either or both of the first cell conductivity information and the second cell conductivity information may be used to generate the TOC level (see below).

In step 206, the first cell conductivity information is compared to the second cell conductivity information in order to determine the presence of interference materials. The difference between the first cell conductivity increase and the second cell conductivity increase can be determined and can be stored, communicated, displayed, or otherwise employed. If interference materials were produced by the oxidization in the first oxidization cell, then the change in the first cell conductivity may be greater than the change in the second cell conductivity, as the interference materials will increase the conductivity of the fluid under test in the first conductivity cell. Consequently, where the first cell conductivity is substantially equal to the second cell conductivity, it can be determined that no interference materials were produced by the oxidization. This information may be important when assessing the results of the TOC level measurement.

In step 207, the first and second cell conductivities are processed in order to quantify the amount of interference materials produced in the first oxidization cell. The conductivity difference between the first oxidization cell and the second oxidization cell maybe used to quantify the amount of interference materials in the fluid under test. The quantifying can comprise characterizing a carbon atom quantity and a non-carbon atom quantity using a ratiometric analysis of the first oxidization cell conductivity and the second oxidization cell conductivity. The quantification may comprise a quantity value or quantity ratio, but may not necessarily include any identification of interference materials or interference material constituents. Alternatively, this step may include introducing a carrier gas into the first oxidization cell that strips or absorbs the produced carbon dioxide. As a result of the carbon dioxide stripping, the first cell conductivity may then reflect a conductivity value that is due only to the interference materials.

Further or additional steps may be performed at this time, such as performing a post-measurement oxidization step in the second oxidization cell to make sure that the second oxidization cell is not contaminated by carbon materials or interference materials in the fluid under test. This can be done to clean the second oxidization cell. The two cells may then be emptied and further tests may be performed.

FIG. 3 shows the TOC fluid sensor 100 according to another embodiment of the invention. Elements in common with FIG. 1 share reference numbers. In this embodiment, the TOC fluid sensor 100 is constructed as a unitary device, including as a microfluidic or nanofluidic device, for example.

The TOC fluid sensor 100 is formed on a substrate 150, such as glass, quartz, silicon, or other suitable material. On top of the substrate 150 is a channel layer 130, such as a silicon dioxide channel layer, for example. A cap 140 is located on the channel layer 130. The substrate 150, the channel layer 130, and the cap 140 can be formed into a unit. This may include by gluing or bonding, welding, sealing materials, held together by a frame or mechanical device(s), or can be constructed by etching or deposition processes.

A channel is built into the channel layer 130 and divided by the gas permeable membrane 106 in order to produce the first oxidization cell 101A and the second oxidization cell 101B. The first oxidization cell 101A and the second oxidization cell 101B are independent and can be independently operated.

One light source 130A is shown on the cap 140, wherein the cap is divided into a light (or UV light) transmissive region 142 and a light (or UV light) opaque region 144. The light source 130A may be located adjacent to or on the light transmissive region 142 of the cap 140, wherein light from the light source 130A is selectively admitted into the interior of the TOC fluid sensor 100.

It should be understood that more than one light source 130 may be used in the TOC fluid sensor 100, as discussed above. Further, the cap 140 may include light segregation structures or features that align with the oxidization chambers in order to illuminate a corresponding chamber of the TOC fluid sensor 100.

The first oxidization cell 101A includes conductivity measurement electrodes 137A, 138A. The second oxidization cell 101B likewise includes conductivity measurement electrodes 137B, 138B. The conductivity measurement electrodes are used to measure conductivity in the respective oxidization cells.

The first oxidization cell 101A includes a temperature sensor electrode pair 162A and a temperature sensor electrode pair 163A. The second oxidization cell 101B likewise includes a temperature sensor electrode pair 162B and a temperature sensor electrode pair 163B. The temperature sensor electrode pairs may be used to measure fluid temperature in the respective oxidization cells.

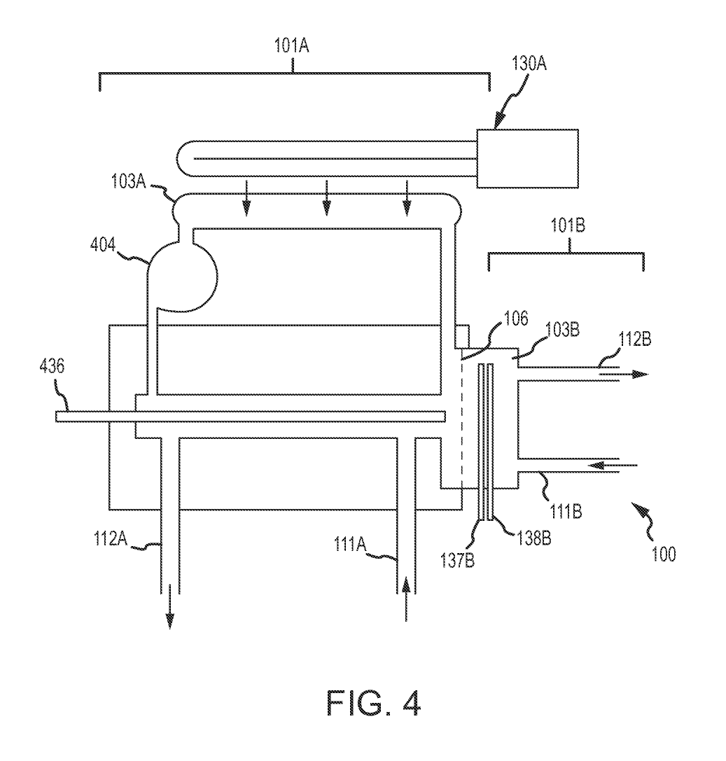

FIG. 4 shows the TOC fluid sensor 100 according to another embodiment of the invention. Elements in common with FIGS. 1 and 3 share reference numbers. In this embodiment, the TOC fluid sensor 100 further includes a pump 404 that is configured to move fluid in the first oxidization cell 101A from the oxidization chamber 103A to the gas permeable membrane 106. Further, the pump 404 may circulate the fluid. The pump 404 allows the membrane 106 to be located away from the oxidizing chamber 103A in order to prevent the membrane 106 from being exposed to the UV light, as the UV light may damage the membrane 106. The circulation can be performed during at least a portion of the oxidizing process. The circulation can be performed during the entire oxidizing process.

The first oxidization cell 101A in this embodiment includes a conductivity sensor (or conductivity electrodes) 436. The conductivity sensor 436 may also be located away from the oxidizing chamber 103A due to the pump 404.

In any of the embodiments shown, the second oxidization cell 101B may comprise a cartridge or replaceable portion. Consequently, the second oxidization cell 101B may include the permeable gas membrane 106 and may include necessary seals and/or attachment features. As a result, the cartridge and membrane 106 can be easily inspected, cleaned, and/or replaced.

Advantageously, where UV light and an oxidizing material are used, the sensor and method can comprise a reagentless oxidization process. The fluid after oxidization does not have any manner of chemical reagent added or present. This beneficially does not present any hazardous or toxic waste disposal problems. In addition, another advantage is that the test is low in cost, safe to perform, and typically does not come under hazardous or toxic materials regulations. However, it should be understood that other suitable oxidization processes may be employed, including oxidization processes that use reagents.

The detailed descriptions of the above embodiments are not exhaustive descriptions of all embodiments contemplated by the inventors to be within the scope of the invention. Indeed, persons skilled in the art will recognize that certain elements of the above-described embodiments may variously be combined or eliminated to create further embodiments, and such further embodiments fall within the scope and teachings of the invention. It will also be apparent to those of ordinary skill in the art that the above-described embodiments may be combined in whole or in part to create additional embodiments within the scope and teachings of the invention. Accordingly, the scope of the invention should be determined from the following claims.

1. A total organic carbon (TOC) fluid sensor, comprising:
a first oxidization cell and a second oxidization cell that receive a fluid under test;
a gas permeable membrane configured to allow carbon dioxide to equilibriate between the first oxidization cell and the second oxidization cell;
a first conductivity sensor configured to measure a first oxidization cell conductivity; and
a second conductivity sensor configured to measure a second oxidization cell conductivity;
wherein the TOC fluid sensor is configured to oxidize a fluid portion to produce carbon dioxide gas, equilibriate the carbon dioxide gas between the first oxidization cell and the second oxidization cell, obtain a second cell conductivity information, and determine a TOC quantity in the fluid under test from the second cell conductivity information when the first cell oxidization is substantially complete.

2. The TOC fluid sensor of claim 1, with the second oxidization cell being substantially free of interference materials that may exist in the first oxidization cell as a result of the equilibriation.

3. The TOC fluid sensor of claim 1, with determining the TOC quantity further comprising determining the TOC quantity from a first cell conductivity information and the second cell conductivity information when the first cell oxidization is substantially complete.

4. The TOC fluid sensor of claim 1, wherein the first cell oxidization is substantially complete when the first cell conductivity information becomes substantially unchanging.

5. The TOC fluid sensor of claim 1, wherein the first cell oxidization is substantially complete when the first cell conductivity information and the second cell conductivity information both become substantially unchanging.

6. The TOC fluid sensor of claim 1, wherein the oxidization can be performed in both the first oxidization cell and in the second oxidization cell in the absence of any interference materials.

7. The TOC fluid sensor of claim 1, with the TOC fluid sensor being further configured to detect interference materials in the fluid under test if a first cell conductivity increase is greater than a second cell conductivity increase.

8. The TOC fluid sensor of claim 1, with the TOC fluid sensor being further configured to quantify interference materials in the fluid under test using the first cell conductivity information and the second cell conductivity information, with the quantifying characterizing a carbon atom quantity and a non-carbon atom quantity using a ratiometric analysis of the first oxidization cell conductivity and the second oxidization cell conductivity.

9. The TOC fluid sensor of claim 1, with the TOC fluid sensor being further configured to introduce a carrier gas that is substantially void of carbon dioxide into the first oxidization cell, with the carrier gas substantially stripping the carbon dioxide, and quantify interference materials in the fluid under test using the first cell conductivity information.

10. The TOC fluid sensor of claim 1, further comprising a pump that recirculates the fluid in the first oxidization cell.

11. A total organic carbon (TOC) fluid measurement method, comprising:
oxidizing a fluid portion of a fluid under test in a first oxidization cell to create carbon dioxide;
equilibriating the carbon dioxide between the first oxidization cell and a second oxidization cell;
obtaining a first cell conductivity information and a second cell conductivity information; and
determining a TOC quantity in the fluid under test from the second cell conductivity information when the first cell oxidization is substantially complete.

12. The method of claim 11, with determining the TOC quantity further comprising determining the TOC quantity from a first cell conductivity information and the second cell conductivity information when the first cell oxidization is substantially complete.

13. The method of claim 11, wherein the first cell oxidization is substantially complete when the first cell conductivity information becomes substantially unchanging.

14. The method of claim 11, wherein the first cell oxidization is substantially complete when the first cell conductivity information and the second cell conductivity information both become substantially unchanging.

15. The method of claim 11, wherein the oxidization can be performed in both the first oxidization cell and in the second oxidization cell in the absence of any interference materials.

16. The method of claim 11, further comprising detecting interference materials in the fluid under test if a first cell conductivity increase is greater than a second cell conductivity increase.

17. The method of claim 11, further comprising quantifying interference materials in the fluid under test using the first cell conductivity information and the second cell conductivity information, with the quantifying characterizing a carbon atom quantity and a non-carbon atom quantity using a ratiometric analysis of the first oxidization cell conductivity and the second oxidization cell conductivity.

18. The method of claim 11, further comprising:
introducing a carrier gas that is substantially void of carbon dioxide into the first oxidization cell, with the carrier gas substantially stripping the carbon dioxide; and
quantifying interference materials in the fluid under test using the first cell conductivity information.

19. The method of claim 10, further comprising re-circulating the fluid in the first oxidization cell during at least a portion of the oxidizing.
